(19) United States
(12) Patent Application Publication
(10) Pub. No.: US 2022/0313119 A1
(43) Pub. Date: Oct. 6, 2022

(54) ARTIFICIAL INTELLIGENCE-BASED SHOULDER ACTIVITY MONITORING SYSTEM

(71) Applicant: OrthoKinetic Track LLC, Newton, MA (US)

(72) Inventors: Rabeeh Majidi, Newton, MA (US); Ali Kiapour, Newton, MA (US)

(73) Assignee: OrthoKinetic Track LLC, Newton, MA (US)

(21) Appl. No.: 17/708,758

(22) Filed: Mar. 30, 2022

Related U.S. Application Data
(60) Provisional application No. 63/168,675, filed on Mar. 31, 2021.

(51) Int. Cl. A61B 5/11; A61B 5/00; A61B 5/055; G16H 50/20; G16H 10/60; G16H 40/67; G09B 19/00

(52) U.S. Cl. CPC A61B 5/1121; A61B 5/1107; A61B 5/4848; A61B 5/055; A61B 5/7264; G16H 50/20; G16H 10/60; G16H 40/67; G09B 19/003; A61B 2562/0219; G06F 3/011

(57) ABSTRACT

Embodiments of the innovation relate to a shoulder activity analysis device, comprising a controller having a processor and memory, the controller configured to: receive shoulder activity data from a set of sensors of a shoulder activity detection device, the shoulder activity data identifying shoulder range of motion and shoulder muscle activity of a user; apply the shoulder activity data to a shoulder activity analysis model to identify a user shoulder outcome diagnosis; and based upon the user shoulder outcome diagnosis, output a diagnosis notification to at least one of a user device and a clinician device, the diagnosis notification identifying the user shoulder outcome diagnosis.

### RELATED APPLICATIONS

This patent application claims the benefit of U.S. Provisional Application No. 63/168,675, filed on Mar. 31, 2021, entitled “Artificial Intelligence-Based Muscle Activity Monitoring System,” the contents and teachings of which are hereby incorporated by reference in their entirety.

### BACKGROUND

The shoulder is the most mobile joint in the body and, due to its inherent instability, it is highly prone to injury, dislocation, and degenerative diseases. For example, rotator cuff disease is reported in 20-30% of the general population and up to 40-60% of patients with shoulder pain. While many shoulder conditions are treated non-operatively, a growing number of patients undergo surgical procedures and require prolonged rehabilitation to gain their range of motion and function. The outcome of surgical interventions is commonly measured by improvement in shoulder pain, range of motion (ROM), strength, and patient-reported results outcome measures. For example, adhesive capsulitis (“frozen shoulder”) is a common problem after shoulder surgery, particularly rotator cuff surgery due to a period of immobilization to promote healing. As such, timely assessment of ROM and appropriate interventions by physical therapist can be utilized to mitigate stiffness and development of adhesive capsulitis during the postoperative period.

While improvements have been made in developing and standardizing patient-reported outcome measures, physicians typically rely on physical examination to assess patient's ROM and strength, the results of which can be variable and inaccurate. Further, timely assessment of ROM and appropriate interventions by physical therapists are critical to avoid stiffness and development of adhesive capsulitis during the postoperative period.

With recent technological advancements, real-time motion and muscle activity tracking through a computer-aided medical device has become available. For example, conventional ROM detection devices, such as mobile-based platforms, are used to measure a subject's joint parameters as the subject exercises in front of a camera to capture actual ROM.

### SUMMARY

Conventional ROM detection devices can suffer from a variety of deficiencies. For example as provided above, certain ROM detection devices are utilized to capture a subject's ROM as the subject exercises in front of a camera. However, these conventional devices typically do not include sensors to capture actual ROM and muscle activation. Further, conventional ROM detection devices focus on athletics and are primarily mobile-based platforms which do not provide time-sensitive and personalized guidance to the user for adjustment of treatment during post-operative period.

By contrast to conventional ROM detection devices, embodiments of the present innovation relate to an artificial intelligence (AI)-based shoulder activity monitoring system. In one arrangement, the shoulder activity monitoring system includes a shoulder activity detection device having a variety of sensors which measure biometric information, such as substantially real-time motion and muscle activity, of a user's shoulder joint. The shoulder activity monitoring system also includes an AI-based shoulder activity analysis device configured to receive the sensor signals and to provide feedback to the user based on the signals, such as guidelines for adjusting the user's rehabilitation activities and for performing specific exercises, each of which can help the user or patient regain the full function of the shoulder joint. For example, if the user fails to achieve the expected individualized ROM set by the shoulder activity monitoring system, the shoulder activity monitoring system can provide the user with instructions to complete specific exercises and track their compliance and improvement over time. The AI-based shoulder activity analysis device can also be configured to provide data to a clinician, such as a doctor or physical therapist, to diagnose the user's shoulder more accurately.

The shoulder activity detection device can include a triaxial accelerometer-gyroscope and surface electromyography (EMG) sensors attached to the trapezius, infraspinatus, deltoid, and biceps muscles to generate signals relating to different movements of the shoulder (e.g., joint range of motion and muscle activity). The shoulder activity detection device can provide the signals in a wireless format (e.g., Bluetooth Low Energy (BLE) or wireless IEEE 802.15.4 communication interfaces) to a user device executing a shoulder activity application which, in turn can transfer the signals to the shoulder activity analysis device (e.g., healthcare cloud/storage) via network communication (e.g., WiFi or Cellular Network (4G or 5G)) for analysis. The shoulder activity analysis device can be configured with recurrent neural networks (RNNs) to analyze time-series data and to identify a degree of shoulder impairment.

In one arrangement, the shoulder activity monitoring system can include an augmented reality system having an augmented reality display disposed in electrical communication with the user device. The augmented reality system can provide remote, at-home rehabilitation services to the user while tracing tele-visit sessions in compliance with billing standards as in-person visits. During the tele-visit, the user can wear the shoulder activity detection device and perform specific movements instructed by a clinician (e.g., doctor, orthopedic surgeon, or physical therapist) and provided through an avatar shown on the augmented reality display. Further, the user device, as part of the augmented reality system, can provide the clinician with data relating to the motion and muscle firing activity user in the form of an avatar. Additionally, in response to receiving this data from the user device, the shoulder activity analysis device can provide information relating to the user's ROM to the user device and can provide information relating to the user's ROM as well as other details of the user's muscle activity and joint kinematic activity data to the clinician.

Also, the user able to perform exercises using the augmented reality system with guidance from the user device based on the motion data. The system will store history joint motion history data obtained from the user after the surgery. The user can be instructed to perform exercises towards achieving the goal of improving their joint range of motion. If the user fails to achieve the expected individualized ROM set by the shoulder activity analysis device, the user can receive instructions to complete specific exercises and the shoulder activity analysis device can track the user's compliance and improvement over time. The user can use the shoulder activity detection device for a predefined period per day, and the data can be shared with a clinician (e.g., the surgeon and physical therapist) for continuous and remote monitoring. The real-time joint kinematic and muscle activity data provided by the shoulder activity detection device can help the clinician with better information for more accurate decision making about suitable treatment or intervention.

The shoulder activity monitoring system is configured to provide quantitative and reliable ROM and muscle activity monitoring with remote data-sharing capacity, thereby reducing the need for in-person hospital/physical-therapy visits, increasing user engagement through goal-oriented recovery feedback, and lowering medical costs. The shoulder activity monitoring system can depict deviation from normal recovery and give real-time feedback to the user and clinician during postoperative rehabilitation to support physical therapy, improve range of motion (ROM) and optimize pain control. As such, the shoulder activity monitoring system can close the gap among users and clinicians such as orthopedic surgeons and physical therapists.

Embodiments of the innovation relate to a shoulder activity analysis device, comprising a controller having a processor and memory, the controller configured to: receive shoulder activity data from a set of sensors of a shoulder activity detection device, the shoulder activity data identifying shoulder range of motion and shoulder muscle activity of a user; apply the shoulder activity data to a shoulder activity analysis model to identify a user shoulder outcome diagnosis; and based upon the user shoulder outcome diagnosis, output a diagnosis notification to at least one of a user device and a clinician device, the diagnosis notification identifying the user shoulder outcome diagnosis.

Embodiments of the innovation relate to a shoulder activity detection device, comprising: a support material configured to be disposed in proximity to a user shoulder; a spatial positioning sensor coupled to the support material and configured to generate a shoulder range of motion signal; and a set of shoulder muscle activity sensors coupled to the support material and configured to generate shoulder muscle activity signals.

Embodiments of the innovation relate to a shoulder activity monitoring system, comprising a shoulder activity detection device and a shoulder activity analysis device. The shoulder activity detection device, comprises: a support material configured to be disposed in proximity to a user shoulder, a spatial positioning sensor coupled to the support material and configured to generate a shoulder range of motion signal, and a set of shoulder muscle activity sensors coupled to the support material and configured to generate shoulder activity data. The shoulder activity analysis device comprises a controller having a processor and memory, the controller configured to: receive shoulder activity data from the set of sensors of the shoulder activity detection device, the shoulder activity data identifying shoulder range of motion and shoulder muscle activity of a user, apply the shoulder activity data to a shoulder activity analysis model to identify a user shoulder outcome diagnosis, and based upon the user shoulder outcome diagnosis, output a diagnosis notification to at least one of a user device and a clinician device, the diagnosis notification identifying the user shoulder outcome diagnosis.

### BRIEF DESCRIPTION OF THE DRAWINGS

The foregoing and other objects, features and advantages will be apparent from the following description of particular embodiments of the innovation, as illustrated in the accompanying drawings in which like reference characters refer to the same parts throughout the different views. The drawings are not necessarily to scale, emphasis instead being placed upon illustrating the principles of various embodiments of the innovation.

FIG. 1 illustrates a shoulder activity monitoring system, according to one arrangement.

FIG. 2 illustrates a schematic representation of a shoulder activity analysis framework and shoulder activity analysis model of FIG. 1 configured as a recurrent neural network model, according to one arrangement.

FIG. 3 illustrates a front view of a user and a shoulder activity detection device, according to one arrangement.

FIG. 4 illustrates a side view of the user and the shoulder activity detection device of FIG. 3, according to one arrangement

FIG. 5 is a flowchart illustrating the elements performed by the shoulder activity analysis device during operation.

FIG. 6 illustrates a front view of a user and a shoulder activity detection device, according to one arrangement.

FIG. 7 illustrates a shoulder activity monitoring system, according to one arrangement.

FIG. 8 illustrates a shoulder activity detection device having first and second sets of sensors, according to one arrangement.

FIG. 9 illustrates an augmented reality system of the shoulder activity monitoring system, according to one arrangement.

### DETAILED DESCRIPTION

Embodiments of the present innovation relate to an artificial intelligence (AI)-based shoulder activity monitoring system. In one arrangement, the shoulder activity monitoring system includes a shoulder activity detection device having a variety of sensors which measure biometric information, such as substantially real-time motion and muscle activity, of a user's shoulder joint. The shoulder activity monitoring system also includes an AI-based shoulder activity analysis device configured to receive the sensor signals and to provide feedback to the user based on the signals, such as guidelines for adjusting the user's rehabilitation activities and for performing specific exercises, each of which can help the user regain the full function of the shoulder joint. For example, if the user fails to achieve the expected individualized ROM set by the shoulder activity monitoring system, the shoulder activity monitoring system can provide the user with instructions to complete specific exercises and track their compliance and improvement over time. The AI-based shoulder activity analysis device can also be configured to provide data to a clinician, such as a doctor or physical therapist, to diagnose the user's shoulder more accurately.

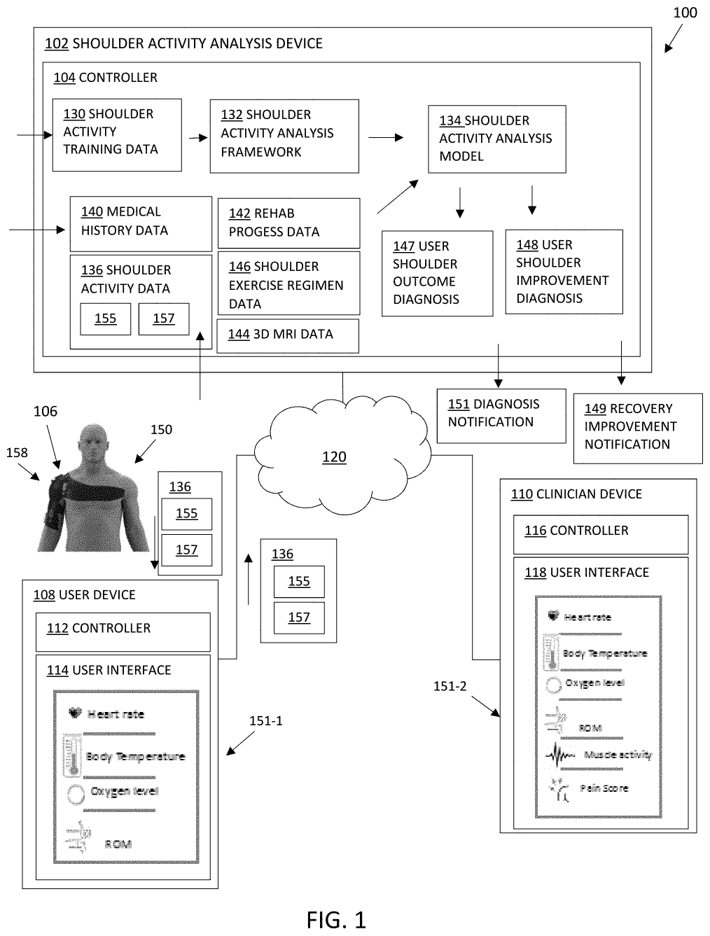

FIG. 1 illustrates a shoulder activity monitoring system 100, according to one arrangement. As illustrated, the shoulder activity monitoring system 100 includes a shoulder activity analysis device 102 disposed in electrical communication with a shoulder activity detection device 106.

The shoulder activity analysis device 102 can be a computerized device having a controller 104, such as a processor and memory. According to one arrangement, the shoulder activity analysis device 102 is disposed in electrical communication with a user device 108 and one or more clinician devices 110 via a network 120, such as a local area network (LAN), a wide area network (WAN), or a public switched telephone network (PSTN). During operation, the shoulder activity analysis device 102 is configured to predict a diagnosis of a user's shoulder, such as following shoulder surgery, based upon sensor data received from the shoulder activity detection device 106. For example, the shoulder activity analysis device 102 is configured to execute a shoulder activity analysis model 134 to predict such a diagnosis and to provide the diagnosis and a recovery notification to the user and clinician devices 108, 110.

To generate the shoulder activity analysis model 134, the shoulder activity analysis device 102 can train a shoulder activity analysis framework 132 using shoulder activity training data 130. For example, the shoulder activity analysis device 102 can retrieve previously-collected training data 130, such as shoulder exercise regimen data and shoulder activity data obtained from multiple users, from a database. The shoulder activity analysis device 102 can apply the shoulder activity training data 130 to the shoulder activity analysis framework 132 to generate the shoulder activity analysis model 134 for use in the system 100.

In one arrangement, the shoulder activity analysis device 102 can continuously develop the shoulder activity analysis model 134 over time. For example, during operation as will be described below, the shoulder activity analysis device 102 can receive shoulder activity data 136 from the shoulder activity detection device 106. The shoulder activity analysis device 102 can be configured to apply the shoulder activity data 136 to the shoulder activity analysis model 134 to further train the model 134. As such, the continuous training of the shoulder activity analysis model 134 can refine or improve the predictive accuracy of the diagnosis and recovery notifications provided to the user over time.

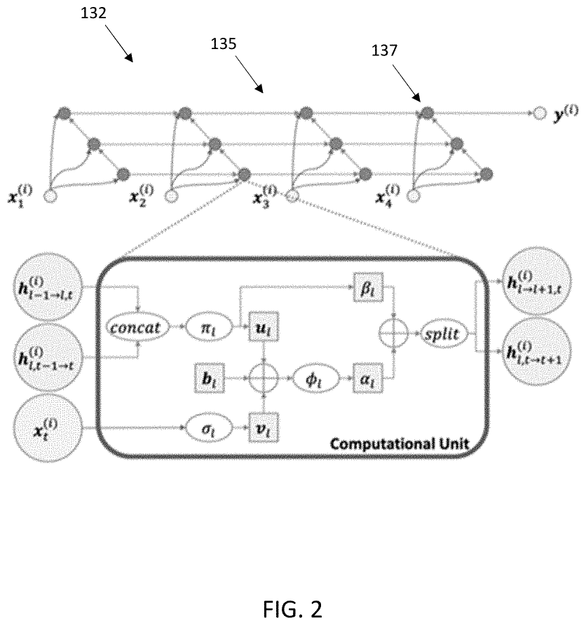

The shoulder activity analysis framework 132 and shoulder activity analysis model 134 can be configured in a variety of ways. In one arrangement, with reference to FIG. 2, the shoulder activity analysis framework 132 and shoulder activity analysis model 134 can be configured as a recurrent neural network model 135, such as a sparse deep recurrent neural network.

For example, as shown in FIG. 2, X₁⁽ⁱ⁾,X₂⁽ⁱ⁾,X₃⁽ⁱ⁾,X₄⁽ⁱ⁾ denote a sequence of four input vectors, such as the sensor data received from four muscle sensors carried by the shoulder activity detection device 106, with four time steps (e.g., seconds) for the i^{th} sample (e.g., user). Further, y₍ᵢ₎ denotes the corresponding label (e.g., binary/multiple predefined disease status), and the nodes 137 denote computational units having internal states (h's) as inputs and outputs, forming three levels in depth.

The recurrent neural network model 135 has relatively strong theoretical training stability which can lead to relatively accurate and robust predictions. For example, the recurrent neural network model 135 is relatively sparse which accounts for a smaller model. The recurrent neural network model 135 can include full-rank transition matrices with only one nonzero value per column, corresponding to either a hidden state or a time-step sample, with the number of parameters equal to the dimensionality of hidden states. Such a matrix is equivalent to a pair of a weight vector (e.g., uₗ, vₗ), and a permutation function (e.g., πₗ, σₗ, ϕₗ), where l∈{1,2,3} denotes the depth index, bₗ denotes a bias term, and αₗ, βₗ denote two scalars for weighted summation. These parameters are shared across time step t∈{1,2,3,4}. In another example, the recurrent neural network model 135 is relatively deep which can compensate for accuracy loss. At each time step, the recurrent neural network model 135 is configured to learn deep structures to approximate highly nonconvex functions with relatively fewer parameters than conventional recurrent neural networks, while achieving similar or even better performance.

Returning to FIG. 1, the shoulder activity detection device 106 is configured to provide shoulder activity data 136 to the shoulder activity analysis device 102. In one arrangement, the shoulder activity detection device 106 can provide shoulder activity data 136 to a user device 108. The user device 108 can be configured as a computerized device, such as a smart phone, tablet, or Personal Data Assistant (PDA) having a controller 112 and user interface 114, such as a display. During operation, the shoulder activity detection device 106 is configured to track the motion and muscle activity of the shoulder of a user 150 and to send the gathered activity as shoulder activity data 136 to the user device 108 through a wireless communication mechanism. The controller 112 of user device 108 is configured to execute a shoulder activity detection application to receive the shoulder activity data 136 and to transmit the shoulder activity data 136 to the shoulder activity analysis device 102 (e.g., a cloud system) via network 120 using a wireless mechanism, such as a WiFi or Cellular Network (e.g., 4G or 5G), for further processing. As such the shoulder activity analysis device 102 is located at a geographically distant location relative to the shoulder activity detection device 106 and user 150 (e.g., in the cloud).

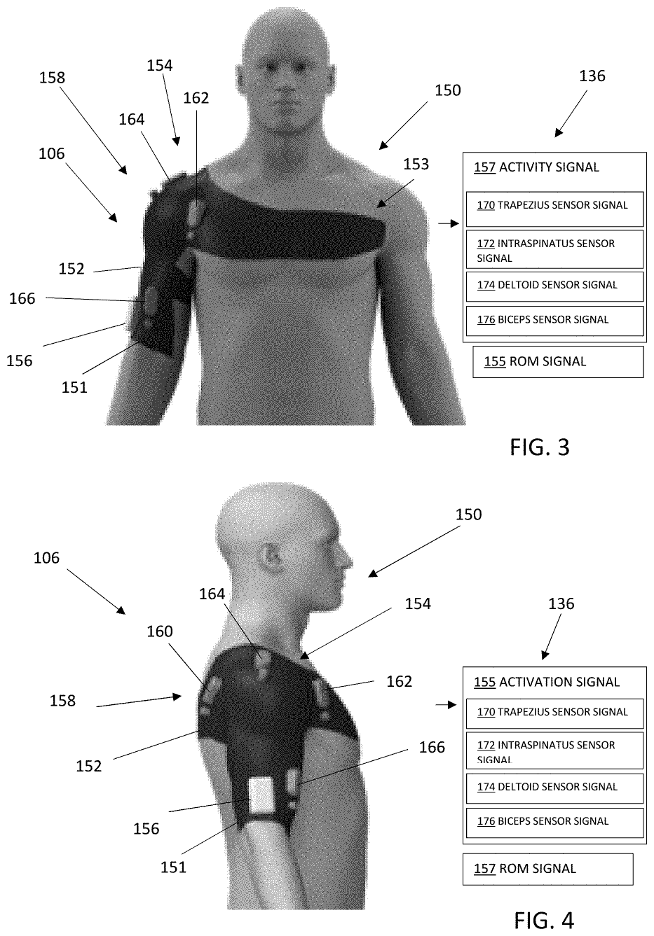

The shoulder activity detection device 106 can be configured in a variety of ways. For example, with reference to FIGS. 3 and 4, the shoulder activity detection device 106 includes a support material 152 configured to be disposed in proximity to a user shoulder 154, a spatial positioning sensor 156 coupled to the support material 152, and a set of shoulder muscle activity sensors 158 coupled to the support material 152.

The support material 152 can be configured as a flexible or elastic material, such as a textile material. With such flexibility, the support material 152 of shoulder activity detection can be configured to mitigate or eliminate discomfort associated with conventional form-fitting wearable devices. Further, the sizing of the support material 152 can be adjustable to fit the user 150 and to conform to the user's shoulder and arm.

The support material 152 can further be configured to account for a variety of environmental factors when utilized by a user 150. For example, shoulder activity detection device 106 can be worn by the user 150 for extended periods of time (e.g., periods ranging from a few minutes to an hour) and can be exposed to moisture from the environment or to sweat from the user 150. As such, the support material 152 can be water resistant or waterproof. Further, the support material 152 can be configured as a medically approved biomaterial or as an antibacterial mesh that provides breathability and minimizes a risk of infection.

The support material 152 can be configured in a variety of geometries. For example, the support material 152 can define a tubular sleeve 151 configured to be disposed over the user's shoulder and arm. In one arrangement, the tubular sleeve 151 can create a compression fit about the user's arm and elbow to retain the positioning of the shoulder activity detection device 106 relative to the user's shoulder during use. In another example, the support material 152 can include a strap portion 153 configured to be disposed about the user's chest to secure the shoulder activity detection device 106 to the user's arm shoulder. In another example, the support material 152 can be manufactured from a single piece of material or can be assembled from modular elements.

The spatial positioning sensor 156 is configured to generate a shoulder range of motion signal 155 in response to motion of the user's shoulder. For example, the spatial positioning sensor 156 can be configured as an accelerometer-gyroscope, such as triaxial accelerometer-gyroscope, coupled to the support material 152 along a line connecting the user's acromion to a lateral epicondyle of the user's humerus near the elbow joint. During operation, as the user 150 moves his or her arm according to a given exercise regimen, the spatial positioning sensor 156 can provide, as part of the shoulder activity data 136, a shoulder range of motion signal 155 to the user device 108 via wireless communication, such as Bluetooth Low Energy (BLE) communication interfaces or an IEEE 802.15.4 wireless system.

The spatial positioning sensor 156 can provide a variety of types of spatial positioning or motion information to the user device 108 as part of the shoulder range of motion signal 155. For example, the spatial positioning sensor 156 can provide ROM data (0-180°) during each performance of the going phase of the exercise regimen (e.g., shoulder abduction, elbow flexion, elbow extension, and shoulder external and internal rotation), ROM data (0-180°) during each performance of the returning phase of the exercise regimen, maximum ROM (degree) for each performance, angular velocity (degree/second) during going phase and returning phase which is the rate of change of angular shoulder displacement or the rate at which shoulder angle is covered in a particular time, average jerk (AJ, m/s3) which is an indicator of movement fluidity such that as the index decreases movement fluidity increase, mean value of the root mean square (RMS) going phase (μV), mean value of the RMS returning phase (μV), muscle contribution (%) for each ROM activity, and RMS graph which is an EMG signal envelope and energy contribution delivered by each muscle in the two phases of the movement (e.g., going and returning phase).

Returning to FIG. 1, the set of shoulder muscle activity sensors 158 are configured to generate shoulder muscle activity signals 157 in response to motion of the user's shoulder. For example, the set of shoulder muscle activity sensors 158 can be to generate interactive functional data relating to the user's muscles including time, duration, and extent of muscle activation during the execution of a given exercise regimen.

With reference to FIGS. 3 and 4, the shoulder activity detection device 106 can include a variety of sensors as part of the set of shoulder muscle activity sensors 158. For example, the shoulder activity detection device 106 can include a trapezius sensor 164 coupled to the support material 152 in a trapezius muscle area of the support material 152, an infraspinatus sensor 160 coupled to the support material 152 in an infraspinatus muscle area of the support material 152, a deltoid sensor 162 coupled to the support material 152 in a deltoid muscle area of the support material 152, and a biceps sensor 166 coupled to the support material 152 in a biceps muscle area of the support material 152. In one arrangement, a position of each of the sensors 160, 162, 164, 166 is adjustable relative to the corresponding muscle areas of the support material 152. For example, a clinician or the user 150 can change the positioning of each of the sensors 160, 162, 164, 166 on the support material 152 to maximize the signal response and accuracy of the signals generated by the sensors 160, 162, 164, 166 during operation. In one arrangement, each of the sensors 158 is configured as a non-invasive sensor, such as a surface electromyography (sEMG) sensor. Further, to maintain contact with a corresponding muscle group during operation, each of the sensors 158 can be configured as a flexible sEMG sensor circuit. Additionally, to mitigate damage caused by exposure to moisture during use, each sensor 158 can be encapsulated in a waterproof material, such as silicone.

Each of the sensors 158 can be disposed in electrical communication with a power source, such as a rechargeable lithium-ion polymer battery to provide over six hours of battery life. Each of the sensors 158 can be low power (e.g., low voltage) to permit extended use between battery charges. In one arrangement, the power supply (e.g., battery) replaceable. Alternately, an integrated power supply which is recharged by wire or wirelessly (e.g., by inductive charging) can be utilized as part of the shoulder activity detection device 106.

The shoulder muscle activity sensors 158 can be disposed on the support material 152 to maximize detection of muscle activity. For example, as illustrated, each of the shoulder muscle activity sensors 158 can be disposed on the same side of the user's body with each sensor 160, 162, 164, 166 having a longitudinal axis aligned substantially parallel to the longitudinal axis of the muscle fibers of the corresponding muscle group (trapezius, infraspinatus, deltoid, and biceps). In one arrangement, each sensor 158 can be configured to measure muscle activity at a rate of about 1000 Hz with sensitivity of 1μV, +/−2% full-scale accuracy and 16 bit resolutions and to generate corresponding shoulder muscle activity signals 157. For example, the trapezius sensor 164 can generate a corresponding trapezius sensor signal 170, the infraspinatus sensor 160 can generate a corresponding infraspinatus sensor signal 172, the deltoid sensor 162 can generate a corresponding deltoid sensor signal 174, and the biceps sensor 166 can generate a corresponding biceps sensor signal 176.

During operation, as the user 150 moves his or her arm according to a given exercise regimen, the shoulder muscle activity sensors 158 can be configured to provide, as part of the shoulder activity data 136, corresponding shoulder muscle activity signals 157 to the user device 108 via wireless communication, such as Bluetooth Low Energy (BLE) communication interfaces or an IEEE 802.15.4 wireless system.

The shoulder activity detection device 106 can include a variety of additional components. In one arrangement, the shoulder activity detection device 106 can include one or more sensors configured to measure parameters of the user's biology. For example, the shoulder activity detection device 106 can include a stretch sensor configured to measure swelling and edema, a joint sensor to detect soft tissue tears such as rotator cuff tears, a pressure sensor configured to measure force applied to the user's shoulder with each movement, a temperature sensor configured to measure the user's temperature, a body oxygen sensor configured to measure the user's oxygen level, and a heart rate monitor configured to measure the user's heart rate. Some individual sensors may perform more than one of the aforementioned functionalities. Groups of sensors may be configured together as one or more sensor arrays. In one arrangement, the shoulder activity detection device 106 can include electronic components such as data storage, battery, and wireless communication antennas (e.g., for WiFi or Cellular Network).

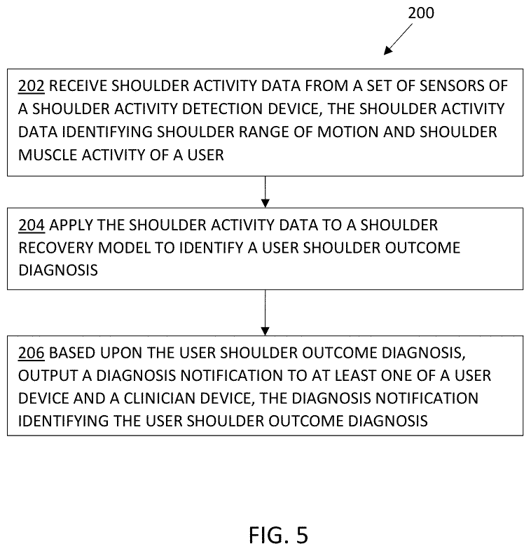

As indicated above, based upon the shoulder activity data 136 received from the shoulder activity detection device 106, the shoulder activity analysis device 102 can be configured to predict a diagnosis of a user's shoulder. FIG. 5 is a flowchart 200 illustrating the elements performed by the shoulder activity analysis device 102 during operation.

In element 202, the shoulder activity analysis device 102 is configured to receive shoulder activity data 136 from a set of sensors 158 of a shoulder activity detection device 106, the shoulder activity data 136 identifying shoulder range of motion and shoulder muscle activity of a user 150.

In one arrangement, with reference to FIG. 1, during operation the user device 108 can guide the user 150 through a given shoulder exercise regimen. The exercise regimen can assist the user 150 in recovering from a shoulder injury or from a shoulder surgical procedure and can cause the set of sensors 158 of the shoulder activity detection device 106 to generate sensor signals. For example, at the start of the exercise regimen, the user 150 can stand upright in a neutral position for 5 to 10 seconds with their arms relaxed by their sides (e.g., palms inward, facing the body). The user device 108 can then receive sensor signals for approximately 10 seconds to establish sensor baseline orientation and noise. Next, the user device 108 can instruct the user to perform an exercise regimen, such as four active and passive movements: shoulder abduction, elbow flexion, elbow extension, and shoulder external and internal rotation. In one arrangement, the exercise regimen can be provided to the user device from the shoulder activity analysis device 102. In one arrangement, a clinician such as a doctor or rehabilitation specialists can send the exercise regimen over the network 120 to the user device 108.

As the user 150 performs the exercise regimen, the sensors 158 of the shoulder activity detection device 106 generate the shoulder activity data 136 as a range of motion signal 155 and as shoulder muscle activity signals 157. For example, during operation of the shoulder activity detection device 106, the spatial positioning sensor 156 of the shoulder activity detection device generates the shoulder range of motion signal 155. Further, with respect to the generation of the shoulder muscle activity signals 157, and with additional reference to FIGS. 3 and 4, the trapezius sensor 164 generates a trapezius sensor signal 170, the infraspinatus sensor 160 generates an infraspinatus sensor signal 172, the deltoid sensor 162 generates a deltoid sensor signal 174, and the biceps sensor 166 generates a biceps sensor signal 176. The sensors 158 transmit the corresponding signals 155, 157 to the user device 108 for transmission to the shoulder activity analysis device 102. The sensors 158 can provide the signals 155, 157 on a variety of timelines, such as continuously,

In one arrangement, prior to providing the shoulder range of motion signal 155 and the shoulder muscle activity signals 157 to the shoulder activity analysis device 102, the user device 108 can apply a processing function to the signals 155, 157. For example, the user device 108 can apply a low pass filter to the shoulder muscle activity signals 157, such as a filter at 100 Hz, to attenuate noise out of the movement frequency band. The user device 108 can also time-synchronized and resampled the signals 155, 157 to a standard rate, such as 200 Hz. For each cyclic motion, the user device 108 can extract features each shoulder muscle activity signal 157 from the upswing/abduction portion of the motion, including average value, maximum value, time to the maximum value, average speed (i.e., rate of change) and maximum speed. The user device 108 can also apply a high-pass filter, such as 10 Hz (fourth-order, Butterworth), to the shoulder muscle activity signals 157 to attenuate motion artifacts and notch filtered to remove 60 Hz interference and its harmonics (second-order IIR filter, notch bandwidth ≤1.5 Hz) and can normalize to the root mean square (RMS) level of each shoulder muscle activity signal 157. Next, to estimate the time-varying standard deviation (a.k.a., “RMS”) of the shoulder muscle activity signals 157, for each signal 170, 172, 174, 176, the user device can whiten each signal via a first backward difference filter rectified and scaled by √2, apply a low-pass filter at 10 Hz, and noise offset-correct each signal via the root difference of squares (RDS) between the computed value and noise calibrated from a rest recording. For each cyclic motion, the user device 108 apply the same processing and data extraction to the shoulder range of motion signal 155.

When the shoulder activity analysis device 102 receives the signals 155, 157 from the user device 108, the shoulder activity analysis device 102 can apply a sensor fusion function to the shoulder range of motion signal 155 and the shoulder muscle activity signals 157. With application of the sensor fusion function, in one arrangement, the shoulder activity analysis device 102 is configured to normalize and combine the signals 155, 157 from the different types of sensors carried by the shoulder activity detection device 106 (e.g., biometric, sEMG's, etc.). In one arrangement, with application of the sensor fusion process, the shoulder activity analysis device 102 is configured to identify which signals 155, 157 are important and to prioritize the signals 155, 157 in order of importance.

Returning to FIG. 5, in element 204, the shoulder activity analysis device 102 is configured to apply the shoulder activity data 136 to a shoulder activity analysis model 134 to identify a user shoulder outcome diagnosis 147. In one arrangement, by applying the processed range of motion signal 155 and shoulder muscle activity signals 157 to the shoulder activity analysis model 134, the shoulder activity analysis device 102 can generate a prediction as to the function of the user's shoulder and can output the prediction as the user shoulder outcome diagnosis 147. For example, based upon the application of the model 134 to the signals 155, 157, the user shoulder outcome diagnosis 147 can identify the probable medical outcome for the user's shoulder (e.g., improving, worsening, etc.) or can identify a potential complication, such as stiffness or frozen shoulder in users or patients with rotator cuff issues.

In one arrangement, the shoulder activity analysis device 102 is configured to apply the shoulder activity data 136 to a shoulder activity analysis model 134 to identify a user shoulder improvement diagnosis 148. For example, based upon the application of the model 134 to the signals 155, 157, the user shoulder improvement diagnosis 148 can include information related to shortening the user's recovery time or to mitigating complications to the user's shoulder.

In one arrangement, the shoulder activity analysis device 102 can apply additional data to the shoulder activity analysis model 134 to predict the user shoulder outcome diagnosis 147 and/or the user shoulder improvement diagnosis 148. For example, with reference to FIG. 1, the shoulder activity analysis device 102 can apply any or all of user medical history data 140, user rehabilitation progress data 142, shoulder exercise regimen data 146, and two-dimensional and/or three-dimensional magnetic resonance imaging data 144 for application to the shoulder activity analysis model 134, in addition to the shoulder activity data 136. The shoulder activity analysis device 102 can receive this data in a variety of ways. For example, the user 150 can provide the medical history data 140 to the shoulder activity analysis device 102 via the user device 108 just prior to starting the exercise regimen. The shoulder activity analysis device 102 can receive the user rehabilitation progress data 142 from the shoulder activity detection device 106 as the user wears the device 106 during the course of a day. The shoulder activity analysis device 102 can receive the shoulder exercise regimen data 146 from a clinician, such as via the clinician device 110, to identify the exercise regimen assigned to the user 150 for rehabilitation. The shoulder activity analysis device 102 can receive the three-dimensional magnetic resonance imaging data 144 as a three-dimensional magnetic resonance image of similar users or patients, such as provided by a healthcare database.

Returning to FIG. 5, in element 206, based upon the user shoulder outcome diagnosis 147, the muscle activity monitoring apparatus 102 is configured to output a diagnosis notification 151 to at least one of a user device 108 and a clinician device 110, the diagnosis notification 151 identifying the user shoulder outcome diagnosis 147. For example, with reference to FIG. 1, the muscle activity monitoring apparatus 102 can output diagnosis notifications 151-1, 151-2 to each of the user device 108 and the clinician device 110. The diagnosis notification 151-1 sent to the user device 108 can identify the probable medical outcome or a potential complication for the user's shoulder. Additionally, the diagnosis notification 151-1 sent to the user device 108 can include data measured by the shoulder activity detection device 106 (e.g., heart rate, temperature, oxygen level, and ROM). The diagnosis notification 151-2 sent to the clinician device 110, such as a device used by a doctor or physical therapist, can include additional information which can be used in further treating the user, such as muscle activity data and a pain score.

In one arrangement, the muscle activity monitoring apparatus 102 is configured to output a recovery improvement notification 149 to the user device 108 and/or the clinician device 110. For example, the recovery improvement notification 149 can include a pain control notification, such as a notification to adjust medication dosing, a suggestion for edema control, or a suggestion for resolving stiffness. In another example, the recovery improvement notification 149 can include a recovery time reduction notification, such as a recommendation to physical therapy, a recommendation to change or adjust the exercise regimen, a recommendation to improving ROM, a recommendation for virtual coaching, a recommendation for an AP based therapy program, or a recommendation for surgery. In another example, the recovery improvement notification 149 can include an advisor consultation notification, such as a notification to consult with doctor or clinician.

In one arrangement, in response to receiving the recovery improvement notification 149 and/or the diagnosis notification 151, the clinician can provide the user 150, via the user device 108, with feedback or information pertaining to the user shoulder improvement diagnosis 148 and/or the user shoulder outcome diagnosis 147. For example, the clinician can provide the user device 108 with an updated rehabilitation program or exercise regimen over the course of the user's use of the shoulder activity detection device 106.

The shoulder activity monitoring system 100 is configured to provide quantitative and reliable ROM and muscle activity monitoring with remote data-sharing capacity via a network 120, thereby reducing the need for in-person hospital/physical-therapy visits, increasing user engagement through goal-oriented recovery feedback, and lowering medical costs. The shoulder activity monitoring system 100 can identify deviation from normal recovery and give real-time feedback to the user 150 and clinician during postoperative rehabilitation to support physical therapy, improve range of motion (ROM), and optimize pain control. As such, the shoulder activity monitoring system 100 can close the gap among users and clinicians such as orthopedic surgeons and physical therapists. Further, shoulder activity monitoring system 100 allows for remote telemedicine delivery of customized rehabilitation services. Additionally, the shoulder activity monitoring system 100 can also be used at clinician office/hospital to help with an initial diagnosis of the user 150.

As indicated above, the shoulder activity monitoring system 100 can be configured to predict a diagnosis of a user's shoulder, such as following shoulder surgery. In one arrangement, the shoulder activity monitoring system 100 is configured to identify the presence of disease in a user's shoulder, such as shoulder stiffness or frozen shoulder. Facioscapulohumeral muscular dystrophy (FSHD) is a genetic debilitating muscular dystrophy with a wide range of disease onset and severity that causes significant impairment of shoulder girdle and proximal arm function and which has no treatment. The shoulder activity monitoring system 100 can be utilized to slow the progress of FSHD by remotely monitoring the user 150.

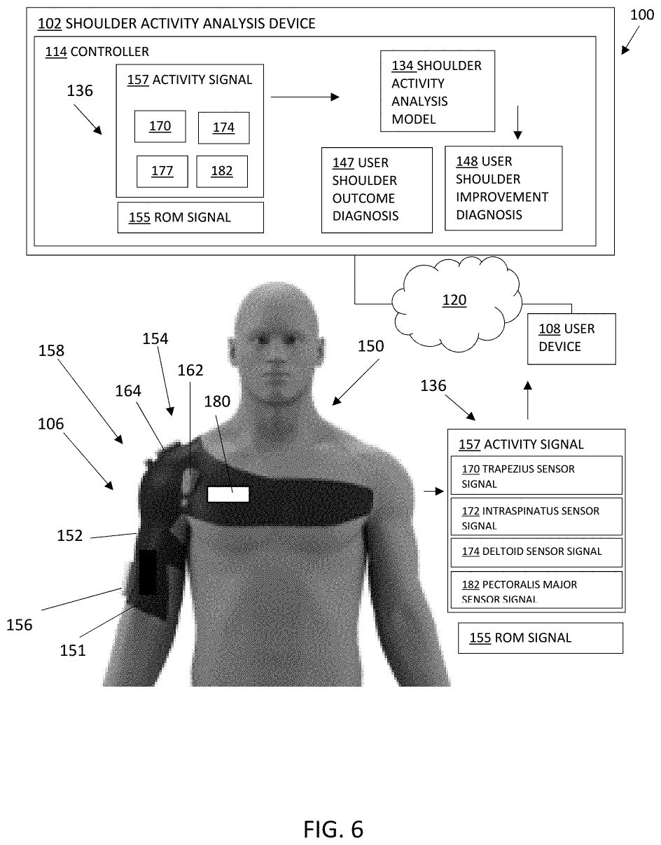

For example, with reference to FIG. 6, the set of sensors 158 of the shoulder activity detection device 106 can include the trapezius sensor 164 (not shown) coupled to the support material 152 in a trapezius muscle area of the support material 152, the infraspinatus sensor 160 coupled to the support material 152 in an infraspinatus muscle area of the support material 152, and the deltoid sensor 162 coupled to the support material 152 in a deltoid muscle area of the support material 152. Further, the set of sensors 158 can also include a pectoralis major sensor 180 coupled to the support material 152 in a pectoralis major muscle area of the support material 152. The pectoralis major muscle includes a sternal portion and a clavicular portion. As such, the pectoralis major sensor 180 is disposed in proximity to both sternal and clavicular portions of the pectoralis major muscle. The pectoralis major sensor 180 can be configured as a non-invasive sensor, such as a surface electromyography (sEMG) sensor or as a flexible sEMG sensor.

During operation, as the user 150 moves his or her arm/elbow according to a given exercise regimen, the shoulder muscle activity sensors 158 can be configured to provide, as part of the shoulder activity data 136, shoulder muscle activity signals 157, such as a trapezius sensor signal 170, an infraspinatus sensor signal 172, a deltoid sensor signal 174, and a pectoralis major sensor signal 182, to the user device 108 via wireless communication, such as Bluetooth Low Energy (BLE) communication interfaces or an IEEE 802.15.4 wireless system. The user device 108 forwards the shoulder activity data 136 to the muscle activity monitoring apparatus 102 which applies the shoulder activity data 136 to the shoulder activity analysis model 134. As a result of such application, the muscle activity monitoring apparatus 102 can generate a user shoulder outcome diagnosis 147 which provides a prediction or warning to the user 150 or clinician regarding potential complications relating to FSHD. Further, as a result of such application, the muscle activity monitoring apparatus 102 can generate a user shoulder improvement diagnosis 148 which provides a potential FSHD recovery plan. With such a configuration, the shoulder activity monitoring system 100 can quantify the degree of shoulder girdle muscle impairment in FSHD patients.

As provided above, the shoulder activity analysis device 102 is configured to execute a shoulder activity analysis model 134 to predict a diagnosis of a user's shoulder, such as following shoulder surgery, based upon sensor data received from the shoulder activity detection device 106 and to provide the diagnosis and a recovery notifications 151, 149 to the user and clinician devices 108, 110. In one arrangement, the shoulder activity analysis model 134 includes separate models related to each of the muscles monitored by the shoulder activity detection device 106.

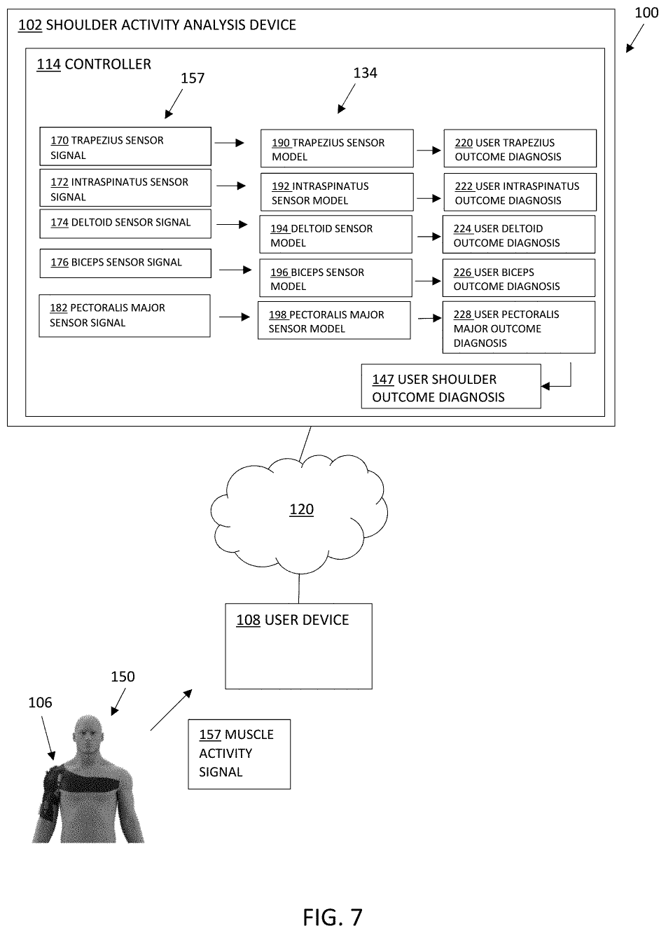

For example, with reference to FIG. 7, the muscle activity monitoring apparatus 102 is configured with a trapezius sensor model 190, an infraspinatus sensor model 192, a deltoid sensor model 194, a biceps sensor model 196, and a pectoralis major sensor model 198. In one arrangement, each model 190, 192, 194, 196, 198 is configured as a recurrent neural network model 135, such as a sparse deep recurrent neural network. During operation, as the user 150 moves his or her arm according to a given exercise regimen, the shoulder muscle activity sensors 158 can be configured to provide, a trapezius sensor signal 170, an infraspinatus sensor signal 172, a deltoid sensor signal 174, and either a biceps sensor signal 176 or a pectoralis major sensor signal 182 to the user device 108.

Upon receipt of these signals 170, 172, 174, 176, 182 from the user device 108, the muscle activity monitoring apparatus 102 applies the signals to a corresponding model to generate an outcome diagnosis. For example, the muscle activity monitoring apparatus 102 can apply the trapezius sensor signal 170 to a trapezius activity analysis model 190 to identify a user trapezius outcome diagnosis 220, the infraspinatus sensor signal 172 to an infraspinatus activity analysis model 192 to identify a user infraspinatus outcome diagnosis 222, the deltoid sensor signal 174 to a deltoid activity analysis model 194 to identify a user deltoid outcome diagnosis 224, the biceps sensor signal 176 to a biceps activity analysis model 196 to identify a user biceps outcome diagnosis 226, and the pectoralis major sensor data 182 to a pectoralis major activity analysis model 198 to identify a user pectoralis major outcome diagnosis 228. The muscle activity monitoring apparatus 102 can then identify a user shoulder outcome diagnosis 147 based upon one or more of the user trapezius outcome diagnosis 220, the user infraspinatus outcome diagnosis 222, the user deltoid outcome diagnosis 224, the user biceps outcome diagnosis 226, and user pectoralis major outcome diagnosis 228. By utilizing separate models 134 for signals pertaining to particular muscles, the muscle activity monitoring apparatus 102 can link clinically relevant pathologies to the mechanics and functions of individual muscles affected by various maladies, such as FSHD, to generate accurate user shoulder outcome diagnoses 147.

As provided above, the shoulder activity analysis device 102 can apply user rehabilitation progress data 142 to the shoulder activity analysis model 134 to generate a user shoulder outcome diagnosis 147. The application of the rehabilitation progress data 142 can be performed in a variety of ways. For example, the shoulder activity analysis device 102 can compare shoulder activity data 136 received from the shoulder activity detection device 106 during an exercise regimen with baseline shoulder activity data to generate user rehabilitation progress data 142. In one arrangement, the shoulder activity detection device 106 is configured to provide the shoulder activity analysis device 102 with initial data to generate the baseline shoulder activity data for a given user 150.

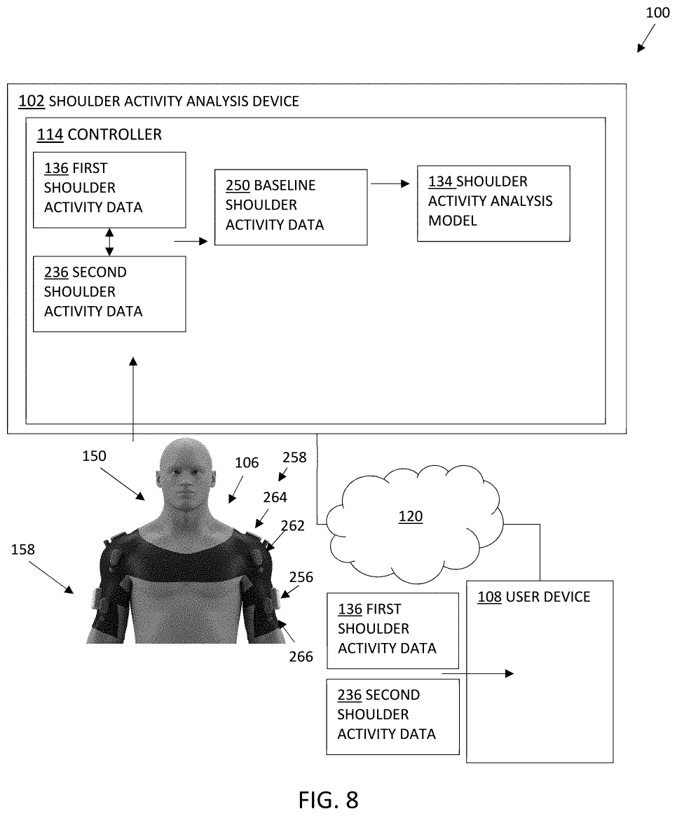

For example, with reference to FIG. 8, the shoulder activity detection device 106 includes a support material 152 that can define a first sleeve 151 for a first user arm and a second sleeve 251 for a second user arm. The first sleeve 151 can include a first set of sensors 158, such as described above. The second sleeve 251 can include a second set of sensors 258 which correspond to the sensors of the first sleeve 151. For example, the second sleeve 251 can include a trapezius sensor 264, an infraspinatus sensor 260 (not shown), a deltoid sensor 262, a biceps sensor 266, and a spatial positioning sensor 256. While not shown, the first sleeve 151 can also include a first pectorals major sensor and the second sleeve 251 can include a second pectorals major sensor.

During operation, as a user performs an initial exercise regimen, the shoulder activity detection device 106 generates first shoulder activity data 136 from the first set of sensors of the first sleeve 151. The first shoulder activity data 136 can identify shoulder range of motion and shoulder muscle activity of a first shoulder of the user 150. For example, the first shoulder activity data 136 can identify shoulder range of motion and shoulder muscle activity of an injured or compromised shoulder of the user 150. Also during operation, as the user performs the initial exercise regimen, the shoulder activity detection device 106 can generate second shoulder activity data 256 from the second set of sensors 258, the second shoulder activity data 256 identifying shoulder range of motion and shoulder muscle activity of a second shoulder of the user. For example, the second shoulder activity data 236 can identify shoulder range of motion and shoulder muscle activity of a healthy or uncompromised shoulder of the user 150.

When the shoulder activity analysis device 102 receives the first shoulder activity data 136 and second shoulder activity data 236, the shoulder activity analysis device 102 can compare the first shoulder activity data 136 and the second shoulder activity data 236 to identify baseline shoulder activity data 250 for one of the first shoulder and the second shoulder of the user. For example, the baseline shoulder activity data 250 can identify the difference between the range of motion and muscle activities of the injured shoulder versus the healthy shoulder.

As provided above, during use of the shoulder activity detection device 106, the user 150 can perform an exercise regimen provided via the user device 108 to increase the range of motion of the user's shoulder over time. In one arrangement, the shoulder activity detection device 106 can include an augmented reality system 300 configured to direct the user to perform the exercise regimen.

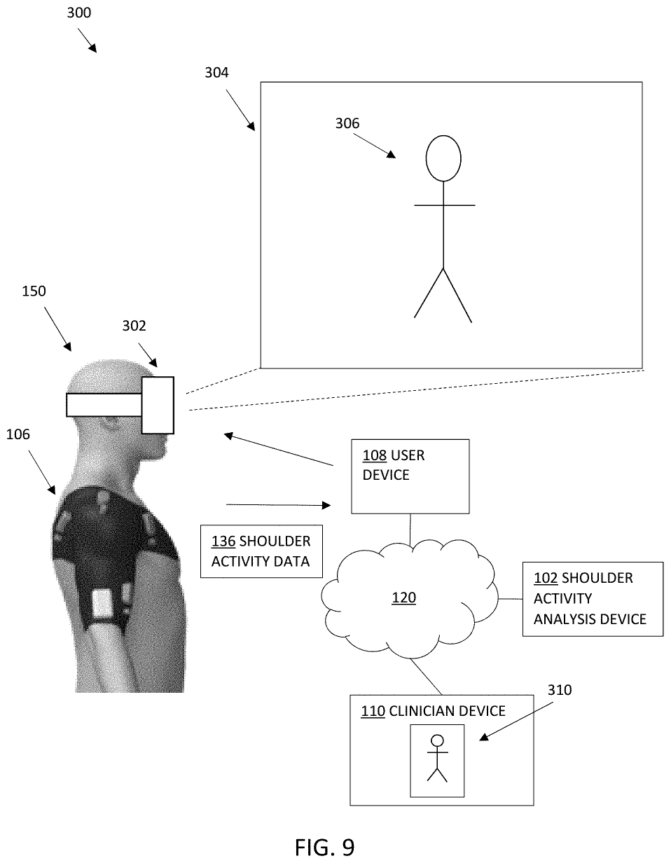

For example, with reference to FIG. 9, the augmented reality system 300 can include an augmented reality display 302 disposed in electrical communication with the user device 108, such as via a wireless connection. The user device 108 is configured to send an image signal to the augmented reality display 302, such as an image of a person or avatar 306 which moves through an exercise regimen, such as prescribed by a clinician. As the augmented reality display 302 shows the moving avatar 306, the avatar 306 can guide the user 150 through a shoulder exercise regimen. As the user mimics the motion of the avatar 306 during the exercise regimen, the shoulder activity detection device 106 can forward shoulder activity data 136 to a clinician device 110 via network 120.

In one arrangement, such as during a tele-visit, the clinician device 110 can receive the shoulder activity data 136 and can display an avatar 310 representing the user 150 and showing the motion and muscle firing activity data detected by the shoulder activity detection device 106. As such, the clinician (e.g., doctor or orthopedic surgeon) can provide feedback (e.g., live and interactive feedback) to adjust the exercise regimen, if needed. Accordingly, the use of the augmented reality system 300 allows for remote at-home telemedicine delivery of rehabilitation services while tracing tele-visit sessions which conform to the standards of in-person visits.

As provided above, the shoulder activity detection device 106 includes a support material 152 configured to be disposed in proximity to a user shoulder 154, a spatial positioning sensor 156 coupled to the support material 152, and a set of shoulder muscle activity sensors 158 coupled to the support material 152. Such description is by way of example only. The support material can be configured to be worn and utilized by a user 150 on any joint, such as a knee, elbow, hip and/or ankle, or on any other body part.

While various embodiments of the innovation have been particularly shown and described, it will be understood by those skilled in the art that various changes in form and details may be made therein without departing from the spirit and scope of the innovation as defined by the appended claims.

What is claimed is:

1. A shoulder activity analysis device, comprising:
a controller having a processor and memory, the controller configured to:
receive shoulder activity data from a set of sensors of a shoulder activity detection device, the shoulder activity data identifying shoulder range of motion and shoulder muscle activity of a user;
apply the shoulder activity data to a shoulder activity analysis model to identify a user shoulder outcome diagnosis; and
based upon the user shoulder outcome diagnosis, output a diagnosis notification to at least one of a user device and a clinician device, the diagnosis notification identifying the user shoulder outcome diagnosis.

2. The shoulder activity analysis device of claim 1, wherein when receiving shoulder activity data from a set of sensors of a shoulder activity monitoring sleeve, the controller is configured to:
receive a trapezius sensor signal from a trapezius sensor of the shoulder activity detection device;
receive an infraspinatus sensor signal from an infraspinatus sensor of the shoulder activity detection device;
receive a deltoid sensor signal from a deltoid sensor of the shoulder activity detection device;
receive one of a biceps sensor signal from a biceps sensor of the shoulder activity detection device and a pectoralis major sensor signal from a pectoralis major sensor; and
receive a shoulder range of motion signal from a spatial positioning sensor of the shoulder activity detection device.

3. The shoulder activity analysis device of claim 2, wherein when applying the shoulder activity data to the shoulder activity analysis model to identify the user shoulder outcome diagnosis, the controller is configured to:
apply the trapezius sensor signal to a trapezius activity analysis model to identify a user trapezius outcome diagnosis;
apply the infraspinatus sensor signal to an infraspinatus activity analysis model to identify a user infraspinatus outcome diagnosis;
apply the deltoid sensor signal to a deltoid activity analysis model to identify a user deltoid outcome diagnosis;
apply one of the biceps sensor signal to a biceps activity analysis model to identify a user biceps outcome diagnosis and the pectoralis major sensor data to a pectoralis major activity analysis model to identify a user pectoralis major outcome diagnosis; and
identify a user shoulder outcome diagnosis based upon at least one of the user trapezius outcome diagnosis, the user infraspinatus outcome diagnosis, the user deltoid outcome diagnosis, and the one of the user biceps outcome diagnosis and user pectoralis major outcome diagnosis.

4. The shoulder activity analysis device of claim 1, wherein the controller is further configured to:
receive first shoulder activity data from a first set of sensors of a first sleeve of the shoulder activity detection device, the first shoulder activity data identifying shoulder range of motion and shoulder muscle activity of a first shoulder of the user;
receive second shoulder activity data from a second set of sensors of a second sleeve of the shoulder activity detection device, the second shoulder activity data identifying shoulder range of motion and shoulder muscle activity of a second shoulder of the user; and
compare the first shoulder activity data and the second shoulder activity data to identify baseline shoulder activity data for one of the first shoulder and the second shoulder of the user.

5. The shoulder activity analysis device of claim 1,
wherein the controller is further configured to receive at least one of user medical history data, user rehabilitation progress data, shoulder exercise regimen data, and three-dimensional magnetic resonance imaging data; and
when applying the shoulder activity data to the shoulder activity analysis model, the controller is configured to apply the at least one of the user medical history data, the user rehabilitation progress data, the shoulder exercise regimen data, and the three-dimensional magnetic resonance imaging data to the shoulder activity analysis model to identify the user shoulder outcome diagnosis.

6. The shoulder activity analysis device of claim 1, wherein the shoulder activity analysis model is configured as a recurrent neural network model.

7. The shoulder activity analysis device of claim 1, wherein the controller is configured to:
apply the shoulder activity data to the shoulder activity analysis model to identify a user shoulder improvement diagnosis; and
based upon the user shoulder improvement diagnosis, output a recovery improvement notification to at least one of the user device and the clinician device.

8. The shoulder activity analysis device of claim 7, wherein the recovery improvement notification comprises at least one of a pain control notification, a recovery time reduction notification, and an advisor consultation notification.

9. A shoulder activity detection device, comprising:
a support material configured to be disposed in proximity to a user shoulder;
a spatial positioning sensor coupled to the support material and configured to generate a shoulder range of motion signal; and
a set of shoulder muscle activity sensors coupled to the support material and configured to generate shoulder muscle activity signals.

10. The shoulder activity detection device of claim 9, wherein the support material defines a tubular sleeve configured to be disposed over a user shoulder and arm.

11. The shoulder activity detection device of claim 9, wherein the spatial positioning sensor comprises an accelerometer-gyroscope.

12. The shoulder activity detection device of claim 9, wherein the set of shoulder muscle activity sensors comprises:
a trapezius sensor coupled to the support material in a trapezius muscle area of the support material;
an infraspinatus sensor coupled to the support material in an infraspinatus muscle area of the support material;
a deltoid sensor coupled to the support material in a deltoid muscle area of the support material; and
one of a biceps sensor coupled to the support material in a biceps muscle area of the support material and a pectoralis major sensor coupled to the support material in a pectoralis major muscle area of the support material.

13. The shoulder activity detection device of claim 9, wherein at least one of the trapezius sensor, the infraspinatus sensor, the deltoid sensor, and the one of the biceps sensor and pectoralis major sensor is configured as a surface electromyography sensor.

14. The shoulder activity detection device of claim 9, wherein at least one of the trapezius sensor, the infraspinatus sensor, the deltoid sensor, and the one of the biceps sensor and pectoralis major sensor is configured as a flexible surface electromyography sensor.

15. The shoulder activity detection device of claim 9, further comprising an augmented reality system, the augmented reality system comprising:
an augmented reality display; and
a user device disposed in electrical communication with the augmented reality display, the augmented reality display configured to display a user image received from the user device, the user image configured to guide a user through a shoulder exercise regimen.

16. A shoulder activity monitoring system, comprising:
a shoulder activity detection device, comprising:
a support material configured to be disposed in proximity to a user shoulder,
a spatial positioning sensor coupled to the support material and configured to generate a shoulder range of motion signal, and
a set of shoulder muscle activity sensors coupled to the support material and configured to generate shoulder activity data; and
a shoulder activity analysis device, comprising a controller having a processor and memory, the controller configured to:
receive shoulder activity data from the set of sensors of the shoulder activity detection device, the shoulder activity data identifying shoulder range of motion and shoulder muscle activity of a user,
apply the shoulder activity data to a shoulder activity analysis model to identify a user shoulder outcome diagnosis, and
based upon the user shoulder outcome diagnosis, output a diagnosis notification to at least one of a user device and a clinician device, the diagnosis notification identifying the user shoulder outcome diagnosis.

17. The shoulder activity monitoring system of claim 16, wherein the set of shoulder muscle activity sensors comprises:
a trapezius sensor coupled to the support material in a trapezius muscle area of the support material;
an infraspinatus sensor coupled to the support material in an infraspinatus muscle area of the support material;
a deltoid sensor coupled to the support material in a deltoid muscle area of the support material; and
one of a biceps sensor coupled to the support material in a biceps muscle area of the support material and a pectoralis major sensor coupled to the support material in a pectoralis major muscle area of the support material.

18. The shoulder activity monitoring system of claim 16, wherein the controller is configured to receive at least one of user medical history data, user rehabilitation progress data, shoulder exercise regimen data, and three-dimensional magnetic resonance imaging data; and
when applying the shoulder activity data to the shoulder activity analysis model, the controller is configured to apply the at least one of the user medical history data, the user rehabilitation progress data, the shoulder exercise regimen data, and the three-dimensional magnetic resonance imaging data to the shoulder activity analysis model to identify the user shoulder outcome diagnosis.

19. The shoulder activity monitoring system of claim 16, wherein the controller is configured to:
apply the shoulder activity data to the shoulder activity analysis model to identify a user shoulder improvement diagnosis; and
based upon the user shoulder improvement diagnosis, output a recovery improvement notification to at least one of the user device and the clinician device.

20. The shoulder activity monitoring system of claim 16, further comprising an augmented reality system, the augmented reality system comprising:
an augmented reality display; and
a user device disposed in electrical communication with the augmented reality display, the augmented reality display configured to display a user image received from the user device, the user image configured to guide a user through a shoulder exercise regimen.
